(19) United States
(12) Patent Application Publication
(10) Pub. No.: US 2022/0204937 A1
(43) Pub. Date: Jun. 30, 2022

(54) Force Stimulation Loading Device and Working Method Thereof

(71) Applicants: THE FIRST PEOPLE'S HOSPITAL OF CHANGZHOU, Changzhou, Jiangsu (CN); HOHAI UNIVERSITY, CHINA, Changzhou, Jiangsu (CN)

(72) Inventors: Yuanping SHI, Changzhou, Jiangsu (CN); Lu ZHENG, Changzhou, Jiangsu (CN); Guanghua LUO, Changzhou, Jiangsu (CN); Xiaolu ZHU, Changzhou, Jiangsu (CN); Zheng WANG, Changzhou, Jiangsu (CN); Xi YIN, Changzhou, Jiangsu (CN); Qiang YIN, Changzhou, Jiangsu (CN)

(21) Appl. No.: 17/607,041

(22) PCT Filed: Apr. 23, 2020
(86) PCT No.: PCT/CN2020/086396

§ 371 Date: Oct. 28, 2021

(30) Foreign Application Priority Data
Sep. 16, 2019 (CN) 201910869033.2

(51) Int. Cl. C12N 5/077; C12M 1/12; C12M 1/42; A61B 90/00

(52) U.S. Cl. CPC C12N 5/0657; C12M 25/02; A61B 2090/066; C12M 23/04; A61B 90/06; C12M 35/04

(57) ABSTRACT

The invention provides a force stimulation loading device and a working method thereof, the force stimulation loading device comprises: a containing body, a stretching mechanism and a twisting mechanism; the containing body is suitable for containing a gel encapsulating cardiomyocytes, and is made of a non-rigid material; the stretching mechanism is suitable for stretching or squeezing the containing body from opposite sides of the containing body to apply a stretching or squeezing force to the gel; the twisting mechanism is suitable for twisting the containing body to apply a twisting stress to the gel; the force stimulation loading device of the present invention is capable of simultaneously applying the stretching or squeezing force and the twisting stress to the gel encapsulating the cardiomyocytes through the stretching mechanism and the twisting mechanism, that is, applying the stretching or squeezing force and the twisting stress to the cardiomyocytes at the same time.

### TECHNICAL FIELD

The invention belongs to the medical-engineering cross-field, especially the biomechanics and mechano-biology fields, and specifically relates to a force stimulus loading device and a working method thereof.

### BACKGROUND ART

Cardiovascular disease is currently the leading cause of human death worldwide, the development of cardiac muscle tissue engineering provides the most potential solution for the treatment of cardiovascular disease. During the occurrence and development of cardiovascular disease, it is closely related to the changes in the cellular force-electrical microenvironment. In the past ten years, with the development of advanced biomaterials and micro-nano biomanufacturing technology, more and more studies have shown that the regulation of cellular force-electrical microenvironment has an effect on the maturation and functionalization of engineered cardiac muscle tissue and regeneration and repair of cardiac muscle tissue. The mechanical microenvironment of cardiomyocytes in vivo will have various effects on the growth and signal transduction of cardiomyocytes, changes in the mechanical microenvironment caused by diseases can also cause abnormal physiological states of cardiomyocytes. Therefore, studying the effect of mechanical microenvironment on cells is also of great significance for exploring basic theories and the diagnosis and treatment of diseases.

The current research on the regulation of cellular mechanical microenvironment is mainly to simulate the mechanical microenvironment of cells under normal physiological or pathological conditions by controlling the hardness or stiffness of two-dimensional or three-dimensional substrate materials; or to regulate stress state of the cells at the microscale by biomimetic mechanics stretching stimulation on scaffold materials that encapsulating cells, thereby promoting the function of the cardiomyocytes. The conventional electrical stimulation is mainly achieved by designing various forms of electrodes to stimulate the cells in a impulse type. Studies have shown that cardiomyocytes inoculated on conductive composite bracket have a significantly improved response to electrical stimulation, and can better conduct the applied electrical signals to promote the synchronized beating function of cardiomyocytes. Therefore, in the process of in vitro culture, by loading the biomimetic force-electrical stimulation to reconstruct cells, the force-electrical microenvironment is beneficial to improve the preparation process and functional simulation of engineered cardiac muscle tissue, wherein the design and method optimization to force signals or force-electrical coupling signal stimulation device are important part to achieve mature engineered cardiac muscle tissue.

The current research work related to cardiac muscle tissue engineering not only focuses on the selection and optimization of scaffold materials and seed cells, also, the promotion of the system technology of engineered cardiac muscle tissue function by regulating cellular force-electrical microenvironment has often become a hot spot for development. At present, in the aspect of improving the function of cardiomyocytes with the help of mechanical and electrical stimulation in vitro, the main concern is the morphology of cardiomyocytes, the expression of functional proteins and genes, and the frequency and intensity of synchronized contraction, etc. The changes in the elastic modulus of cardiac muscle tissue are closely related to the changes in the function of cardiomyocytes, for example, researchers have found that the hardness of the extracellular matrix not only affects the active contraction force in the cardiomyocytes, but also affects the contraction strain and the beat frequency of the cardiomyocytes. Simultaneously, the gene, protein expression and intercellular communication of cardiomyocytes have also been confirmed to be affected by the mechanical microenvironment of cardiac muscle tissue. Cardiomyocytes can sense the static and dynamic mechanical stimulation in the cell microenvironment through the force-sensing ion channels on the cell membrane, activate the electrophysiology and intracellular associated biochemical responses on the cell membrane, thereby realizing the regulation of the structure and function of the cardiomyocytes. In addition, mechanical factors also play an important role in inducing the mesenchymal stem cells to different to cardiomyocytes and to construct cardiac muscle tissue. Stem cells are usually sensitive to force, mechanical stimulation such as tensile and compressive stress, shear stress, and stretch strain can affect the proliferation, skeletal structure and multidirectional differentiation process of stem cells. Wherein, the shear stress generated by fluid flow plays an important role in embryonic development and organ formation, such as the activation and maturation of newborn cardiomyocytes, and the formation of zebrafish embryonic heart.

In the study of force-electrical coupling environment, when the physiological characteristics of cardiomyocytes respond, it usually requires specific excitation application and cell function testing equipment, however, the equipment in the prior art is mostly single type, and in the aspect of cell culture in the gel, there are some technical problems such as uneven mechanical excitation application and difficult to achieve the stretching and twisting stress at the same time.

### SUMMARY OF THE INVENTION

The invention provides a force stimulation loading device and working method thereof.

In order to solve above technical problem, the invention provides a force stimulation loading device, comprising a containing body, a stretching mechanism and a twisting mechanism; the containing body is suitable for containing a gel encapsulating cardiomyocytes, and is made of a non-rigid material; the stretching mechanism is suitable for stretching or squeezing the containing body from opposite sides of the containing body to apply a stretching or squeezing force to the gel; the twisting mechanism is suitable for twisting the containing body to apply a twisting stress to the gel.

Further, the containing body comprises: an upper cover plate and a lower cover plate, and the upper and lower cover plates are connected by a clamping cover; the inner surface of the upper cover plate is provided with multiple first protrusions at intervals; and the inner surface of the lower cover plate is provided with multiple second protrusions at intervals.

Further, the stretching mechanism comprises: screw mechanisms respectively symmetrically arranged on opposite sides of the containing body; the screw mechanism comprises: a screw motor, a transmission shaft, a screw, and a nut; wherein the screw passes through the nut, and one end thereof is connected to the screw motor through the transmission shaft; the other end of the screw is connected to the clamping cover; each screw motor is adapted to drive the corresponding screw to move away from or toward the clamping cover to stretch or squeeze the gel from opposite sides of the gel.

Further, each nut is arranged on a bracket respectively.

Further, the twisting mechanism comprising a twisting motor and twisting components is arranged on the upper cover plate by an upper clamping plate; wherein the twisting components comprise: a housing, a sun gear, multiple planetary gears meshed with the sun gear, and peripheral rims meshed with the planetary gears; one output shaft of the twisting motor is connected to the sun gear; the peripheral rims are fixed on the upper clamping plate; one gear shaft of the sun gear and gear shafts of the planetary gears are fixed on the housing, and the housing is fixedly connected to a frame through connecting rods; the twisting motor is adapted to drive the sun gear to drive the planetary gears to rotate, and to drive the peripheral rims to rotate, thereby driving the upper cover plate to rotate through the upper clamping plate, and applying a twisting stress to the gel.

Further, the twisting mechanism comprising a twisting motor and twisting components is arranged on the upper cover plate by an upper clamping plate; wherein the twisting components comprise: a housing, a sun gear, multiple planetary gears meshed with the sun gear, and peripheral rims meshed with the planetary gears; one output shaft of the twisting motor is connected to the sun gear; the planetary gears are fixed on the upper clamping plate; one gear shaft of the sun gear and peripheral rims are fixed on the housing, and the housing is fixedly connected to a frame through connecting rods; the twisting motor is adapted to drive the sun gear to drive the planetary gears to rotate, thereby driving the upper cover plate to rotate through the upper clamping plate, and applying a twisting stress to the gel.

Further, the twisting motor is arranged on a support component, the support component comprises: a transverse rod and bearing rods arranged on both ends of the transverse rod.

Further, a lower clamping plate is provided lower of the lower cover plate.

On the other hand, the invention provides a working method of a force stimulation loading device, comprising: stretching or squeezing the containing body from opposite sides of the containing body by the stretching mechanism, to apply a stretching or squeezing force to the gel in the containing body; and twisting the containing body by the twisting mechanism to apply a twisting stress to the gel in the containing body.

The invention has the following advantageous effects: the force stimulation loading device of the invention is capable of simultaneously applying the stretching or squeezing force and the twisting stress to the gel encapsulating the cardiomyocytes through the stretching mechanism and the twisting mechanism, that is, applying the stretching or squeezing force and the twisting stress to the cardiomyocytes at the same time; the invention also facilitates the adhesion of the gel with the upper and lower cover plates respectively by the cooperation between the first protrusions on the upper cover plate and the second protrusions on the lower cover plate, and can greatly reduce sliding and offset of the gel, thereby ensuring that the force can be applied evenly to the gel, that is, evenly applied to the cardiomyocytes.

### BRIEF DESCRIPTION OF ACCOMPANYING DRAWINGS

The invention is further described below in combination with accompanying drawings and embodiments.

FIG. 1 shows the structure of the force stimulation loading device in the embodiment of the invention (omitting part of support components);

FIG. 2 shows the structure of the force stimulation loading device in the embodiment of the invention from another perspective (omitting stretching mechanism);

FIG. 3 shows the structure of the twisting components (omitting housing) of the force stimulation loading device in the embodiment of the invention;

FIG. 4 shows the twisting state of the force stimulation loading device in the embodiment of the invention.

FIG. 5 shows the top view of the structure in which the housing of the twisting component of the embodiment in the invention is fixedly connected with the frame linkages through the connecting rod.

wherein:
1 refers to upper cover plate, 11 refers to first protrusions, 12 refers to rotation center, 13 refers to clamping cover, 2 refers to gel, 21 refers to cardiomyocytes, 3 refers to lower cover plate, 31 refers to second protrusions, 40, 50 refer to screw mechanism, 41, 51 refer to transmission shaft, 42, 52 refer to screw, 43, 53 refer to bracket, 60 refers to lower clamping plate, 70 refers to upper clamping plate, 80 refers to twisting components, 801 refers to housing of twisting components, 81 refers to sun gear, 82 refers to planetary gears, 83 refers to peripheral rims, 84 refers to twisting motor, 91 refers to bearing rod, 92 refers to transverse rod, 93 refers to bearing rod, 931, 932, 933 and 934 refer to connecting rods, 921, 922, 923 and 924 refer to frame linkages.

### SPECIFIC EMBODIMENTS OF THE INVENTION

The structure of the invention is further described in detail with the accompanying drawings.

### Embodiment 1

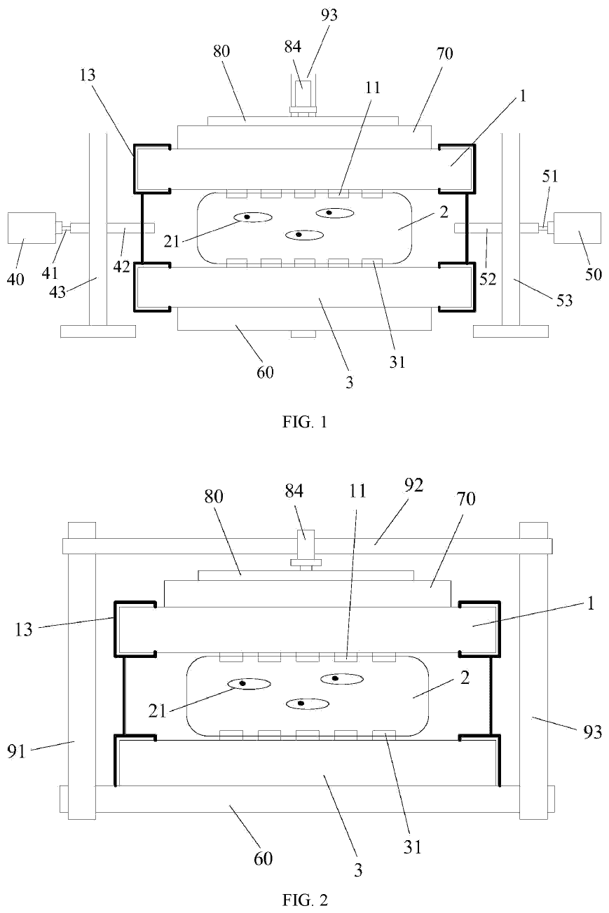

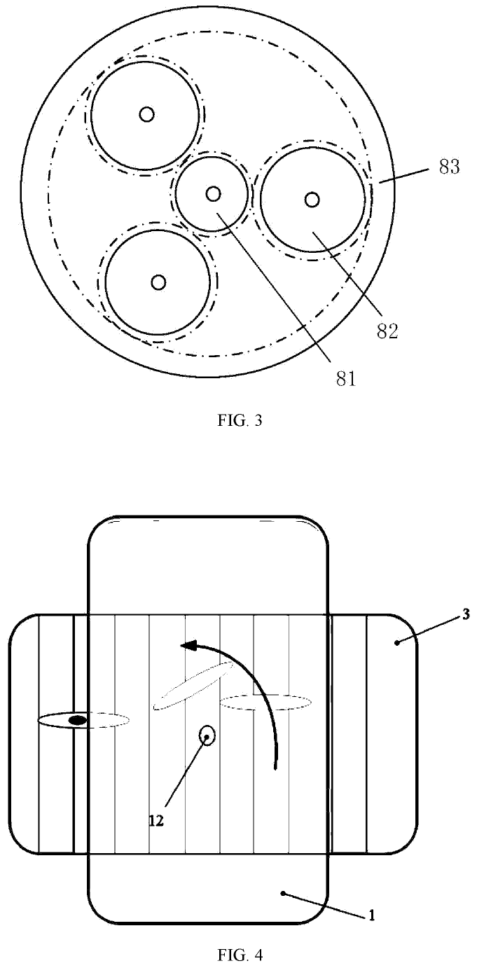

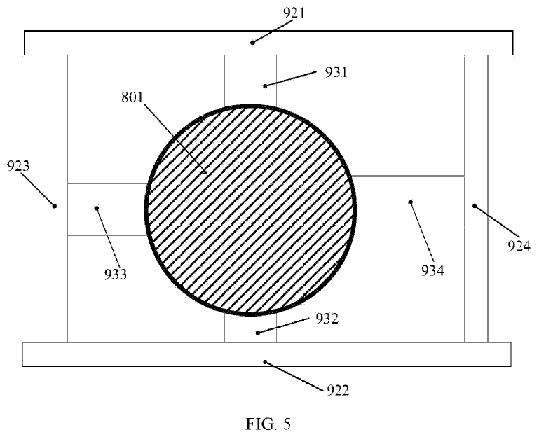

As shown in FIG. 1-5, the embodiment 1 provides a force stimulation loading device, comprising a containing body, a stretching mechanism and a twisting mechanism; the containing body is suitable for containing a gel 2 encapsulting cardiomyocytes 21, and is made of a non-rigid material; the stretching mechanism is suitable for stretching or squeezing the containing body from opposite sides of the containing body to apply a stretching or squeezing force to the gel 2; the twisting mechanism is suitable for twisting the containing body to apply a twisting stress to the gel 2.

Specifically, the force stimulation loading device in the embodiment is capable of simultaneously applying the stretching or squeezing force and the twisting stress to the gel 2 encapsulating the cardiomyocytes 21 through the stretching mechanism and the twisting mechanism.

Further, the containing body comprises: an upper cover plate 1 and a lower cover plate 3, and the upper and lower cover plates are connected by a clamping cover 13; the inner surface of the upper cover plate 1 is provided with multiple first protrusions 11 at intervals; and the inner surface of the lower cover plate 3 is provided with multiple second protrusions 31 at intervals.

Specifically, the materials of the upper cover plate 1 and the lower cover plate are, for example, but not limited to, polydimethylsiloxane (pdms) or polytetrafluoroethylene; the materials of the clamping cover 13 are, for example, but not limited to, polydimethylsiloxane (pdms) or polytetrafluoroethylene; The first protrusions 11 are, for example, but not limited to, rectangular teeth; the second protrusions 31 are also, for example, but not limited to, rectangular teeth; the gel 2 is clamped between the first and second protrusions and facilitates the adhesion of the gel 2 with the upper and lower cover plates respectively by the cooperation between the first protrusions and the second protrusions, and can greatly reduce sliding and offset of the gel, thereby ensuring that the force can be applied evenly to the gel.

Further, the stretching mechanism comprises: screw mechanisms respectively symmetrically arranged on opposite sides of the containing body; the screw mechanism comprises: a screw motor (40; 50), a transmission shaft (41; 51), a screw (42; 52), and a nut; wherein the screw (42; 52) passes through the nut, and one end thereof is connected to the screw motor (40; 50) through the transmission shaft (41; 51); the other end of the screw (42; 52) is connected to the clamping cover 13; each screw motor (40; 50) is adapted to drive the corresponding screw (42; 52) to move away from or toward the clamping cover 13 to stretch or squeeze the gel 2 from opposite sides of the gel 2.

Specifically, the screw mechanisms adopt micro screw mechanism and is controlled by a controlling module; the screw motors (40; 50) adopt micro serve motor to improve the precision of stretching or squeezing; each screw motor (40; 50) is adapted to drive the corresponding screw to move away from or toward the clamping cover 13 to stretch or squeeze the gel 2 from opposite sides of the gel 2, and thereby further ensuring that the stretching or squeezing force can be applied evenly to the gel.

Further, each nut is arranged on a bracket (43; 53) respectively.

As the first embodiment of the twisting mechanism in the embodiment:

the twisting mechanism comprising a twisting motor 84 and twisting components 80 is arranged on the upper cover plate 1 by an upper clamping plate 70; wherein the twisting components 80 comprise: a housing 801, a sun gear 81, multiple planetary gears 82 meshed with the sun gear 81, and peripheral rims 83 meshed with the planetary gears 82; one output shaft of the twisting motor 84 is connected to the sun gear 81; the peripheral rims 83 are fixed on the upper clamping plate 70; one gear shaft of the sun gear 81 and gear shafts of the planetary gears 82 are fixed on the housing 801, the twisting motor 84 is adapted to drive the sun gear 81 to drive the planetary gears 82 to rotate, and to drive the peripheral rims 83 to rotate, thereby driving the upper cover plate 1 to rotate through the upper clamping plate 70, and applying a twisting stress to the gel 2.

Specifically, by fixing the peripheral rims 83 on the upper clamping plate 70, the rotation of the peripheral rims 83 drives the upper cover plate 1 to rotate to apply a twisting stress to the gel 2, the rotation diameter of the embodiment is larger.

Further, the housing 801 is also fixedly connected to the corresponding frame linkages on the frame through connecting rod 931, connecting rod 932, connecting rod 933, and connecting rod 934, that is, connecting rod 931 and frame linkage 921 are fixedly connected, connecting rod 932 and frame linkage 922 are fixedly connected, connecting rod 933 and frame linkage 923 are fixedly connected, and connecting rod 934 and frame linkage 924 are fixedly connected.

As the second embodiment of the twisting mechanism in the embodiment:

the twisting mechanism comprising a twisting motor 84 and twisting components 80 is arranged on the upper cover plate 1 by an upper clamping plate 70; wherein the twisting components 80 comprise: a housing 801, a sun gear 81, multiple planetary gears 82 meshed with the sun gear 81, and peripheral rims 83 meshed with the planetary gears 82; one output shaft of the twisting motor 84 is connected to the sun gear 81; the planetary gears 82 are fixed on the upper clamping plate 70; one gear shaft of the sun gear 81 and peripheral rims 83 are fixed on the housing 801; the twisting motor 84 is adapted to drive the sun gear 81 to drive the planetary gears 82 to rotate, and to drive the peripheral rims 83 to rotate, thereby driving the upper cover plate 1 to rotate through the upper clamping plate 70, and applying a twisting stress to the gel 2.

Specifically, by fixing each planetary gear 82 on the upper clamping plate 70, the rotation of each planetary gear 82 drives the upper cover plate 1 to rotate, thereby applying a twisting stress to the gel 2, the rotation diameter of the embodiment is small.

Further, the housing 801 is also fixedly connected to the corresponding frame linkages on the frame through connecting rod 931, connecting rod 932, connecting rod 933, and connecting rod 934, that is, connecting rod 931 and frame linkage 921 are fixedly connected, connecting rod 932 and frame linkage 922 are fixedly connected, connecting rod 933 and frame linkage 923 are fixedly connected, and connecting rod 934 and frame linkage 924 are fixedly connected.

In practical applications, a suitable twisting mechanism is selected according to the size of the biological sample and the force required to be loaded.

Specifically, the twisting mechanism is also controlled by the controlling module; the gel 2 s twisted around the rotation center 12, the twisting motor 84 adopts micro serve motor to improve the precision of twisting;

Further, the twisting motor 84 is arranged on a support component; the support component comprises: a transverse rod 92 and bearing rods (91;93) arranged on both ends of the transverse rod 92.

Further, a lower clamping plate 60 is provided lower of the lower cover plate 3.

### Embodiment 2

On the basis of Embodiment 1, the Embodiment 2 provides a working method of a force stimulation loading device, comprising: stretching or squeezing the containing body from opposite sides of the containing body by the stretching mechanism, to apply a stretching or squeezing force to the gel; and twisting the containing body by the twisting mechanism to apply a twisting stress to the gel.

Specifically, the specific structure and principle of the force stimulation loading device can refer to the description of Embodiment 1, which will not be repeated here.

In conclusion, the force stimulation loading device can simultaneously apply stretching or squeezing force and the twisting stress on the gel encapsulating cardiomyocytes through stretching mechanism and twisting mechanism, that is, it can simultaneously apply stretching or squeezing force and the twisting stress on cardiomyocytes. In addition, the invention facilitates the adhesion of the gel with the upper and lower cover plates respectively by the cooperation between the first protrusions on the upper cover plate and the second protrusions on the lower cover plate, and can greatly reduce sliding and offset of the gel, thereby ensuring that the force can be applied evenly to the gel, that is, evenly applied to the cardiomyocytes.

Various changes and modifications, inspired by above ideal embodiments according to the invention, without deviating from the technical idea of the invention and according to the above specification, can be made by those skilled in the art. The technical scope of the invention is not limited to the contents of the specification but must be determined according to the scope of claims.

1. A force stimulation loading device, comprising a containing body, a stretching mechanism and a twisting mechanism; the containing body is suitable for containing a gel encapsulating cardiomyocytes, and is made of a non-rigid material; the stretching mechanism is suitable for stretching or squeezing the containing body from opposite sides of the containing body to apply a stretching or squeezing force to the gel; the twisting mechanism is suitable for twisting the containing body to apply a twisting stress to the gel.

2. The force stimulation loading device of claim 1, wherein the containing body comprises: an upper cover plate and a lower cover plate, and the upper and lower cover plates are connected by a clamping cover; the inner surface of the upper cover plate is provided with multiple first protrusions at intervals; and the inner surface of the lower cover plate is provided with multiple second protrusions at intervals.

3. The force stimulation loading device of claim 2, wherein the stretching mechanism comprises: screw mechanisms respectively symmetrically arranged on opposite sides of the containing body; the screw mechanism comprises: a screw motor, a transmission shaft, a screw, and a nut; wherein the screw passes through the nut, and one end thereof is connected to the screw motor through the transmission shaft; the other end of the screw is connected to the clamping cover; each screw motor is adapted to drive the corresponding screw to move away from or toward the clamping cover to stretch or squeeze the gel from opposite sides of the gel.

4. The force stimulation loading device of claim 3, wherein each nut is arranged on a bracket respectively.

5. The force stimulation loading device of claim 2, wherein the twisting mechanism comprising a twisting motor and twisting components is arranged on the upper cover plate by an upper clamping plate; wherein the twisting components comprise: a housing, a sun gear, multiple planetary gears meshed with the sun gear, and peripheral rims meshed with the planetary gears; one output shaft of the twisting motor is connected to the sun gear; the peripheral rims are fixed on the upper clamping plate; one gear shaft of the sun gear and gear shafts of the planetary gears are fixed on the housing, and the housing is fixedly connected to a frame through connecting rods; the twisting motor is adapted to drive the sun gear to drive the planetary gears to rotate, and to drive the peripheral rims to rotate, thereby driving the upper cover plate to rotate through the upper clamping plate, and applying a twisting stress to the gel.

6. The force stimulation loading device of claim 2, wherein the twisting mechanism comprising a twisting motor and twisting components is arranged on the upper cover plate by an upper clamping plate; wherein the twisting components comprise: a housing, a sun gear, multiple planetary gears meshed with the sun gear, and peripheral rims meshed with the planetary gears; one output shaft of the twisting motor is connected to the sun gear; the planetary gears are fixed on the upper clamping plate; one gear shaft of the sun gear and peripheral rims are fixed on the housing, and the housing is fixedly connected to a frame through connecting rods; the twisting motor is adapted to drive the sun gear to drive the planetary gears to rotate, thereby driving the upper cover plate to rotate through the upper clamping plate, and applying a twisting stress to the gel.

7. The force stimulation loading device of claim 5 or 6, wherein the twisting motor is arranged on a support component, the support component comprises: a transverse rod and bearing rods arranged on both ends of the transverse rod.

8. The force stimulation loading device of claim 6, wherein the twisting motor is arranged on a support component, the support component comprises: a transverse rod and bearing rods arranged on both ends of the transverse rod.

9. The force stimulation loading device of claim 2, a lower clamping plate is provided lower of the lower cover plate.

10. A working method of a force stimulation loading device, comprising: stretching or squeezing the containing body from opposite sides of the containing body by the stretching mechanism, to apply a stretching or squeezing force to the gel in the containing body; and twisting the containing body by the twisting mechanism to apply a twisting stress to the gel in the containing body.
